(19) United States
(12) Patent Application Publication
(10) Pub. No.: US 2004/0247633 A1
(43) Pub. Date: Dec. 9, 2004

(54) Novel dermatological composition

(75) Inventors: James J. Eberl, Hilton Head Island, SC (US); Donna Eberl, Hilton Head Island, SC (US); Robert Goldman, New York, NY (US)

Correspondence Address: Henry D. Coleman, 714 Colorado Avenue, Bridgeport, CT, 06605-1601, US

(73) Assignee: EBERSYTES, LLC, Hilton Head Island, SC

(21) Appl. No.: 10/860,726

(22) Filed: Jun. 3, 2004

Related U.S. Application Data
(60) Provisional application No. 60/475,839, filed on Jun. 4, 2003.

(51) Int. Cl. A61K 33/40; A61K 31/555; A61K 31/295; A61K 31/30

(52) U.S. Cl. 424/401; 424/616; 514/184; 514/499; 514/502

(57) ABSTRACT

The invention provides novel dermatological compositions and related methods useful in the activation of skin growth factors and growth receptors. Compositions of the invention act upon follicle cells and other skin targets to induce hair growth, facilitate dermal cell repair, and enhance skin health.

### RELATED APPLICATIONS

This application claims priority from United States provisional application 60/475,829 of same title filed Jun. 4, 2003, the entire contents of which are incorporated by reference herein.

### FIELD OF THE INVENTION

The invention relates to novel dermatological compositions and related methods useful in the activation of skin growth factors and growth receptors. Compositions of the invention act upon follicle cells and other skin targets to induce hair growth, facilitate dermal cell repair, and enhance skin health.

### BACKGROUND OF THE INVENTION

Free radical formation has long been associated with detrimental physical occurrences including tissue damage. Contrary to conventional clinical experience, the instant invention utilizes free radical formation to achieve significant enhancement of dermatological health. While transition metal-containing compositions and enzymes have been used in the treatment of skin disorders and in hair growth stimulation, such compositions have not utilized controlled redox reactions to achieve desired dermatological clinical endpoints.

For example, U.S. Pat. No. 5,888,522 discloses peptone digests complexed with one or more ionic transition metals, such as copper, indium, tin, zinc, or the salts thereof, that are alleged to be useful in treating a variety of skin disorders. Japanese Patent No. 2002332217 to Fujii, et al. discloses hair stimulating compositions containing coenzyme Q.

U.S. Pat. No. 6,544,531 discloses that: (1) retinol or vitamin A alcohol is useful in the reduction of fine lines, wrinkles, and mottled hyperpigmentation in skin; (2) hydroxy acids, and particularly alpha-hydroxy acids, are useful in increasing the clarity of the skin surface, increasing cellular turnover, and increasing skin radiance and smoothness; and (3) ascorbic acid has skin permeation and collagen synthesis activity. U.S. Pat. No. 6,544,531 discloses compositions which include: a retinoid and preferably retinol; a dermatologically active acid; and a volatile base, such as, for example, ammonium hydroxide.

The need continues to exist for improved skin care compositions useful in the treatment of skin disorders and in promotion of hair growth that utilize controlled free radical formation and/or chemical irritants to achieve desired dermatological clinical endpoints such as dermal cell repair or follicle stimulation.

### OBJECTS OF THE INVENTION

It is an object of the invention to provide improved skin care compositions useful in the treatment of skin, skin disorders and in promotion of hair growth.

It is an additional object of the invention to provide improved skin care compositions useful in the treatment of skin disorders and in promotion of hair growth that utilize controlled free radical formation and/or mild chemical irritant to achieve desired dermatological clinical endpoints.

It is a still further object of the invention to provide improved skin care compositions useful in the treatment of skin disorders and in promotion of hair growth that utilize controlled free radical formation and/or mild chemical irritant to achieve dermal cell repair and follicle stimulation.

### SUMMARY OF THE INVENTION

In accordance with the above stated objects, the instant invention provides improved skin care compositions useful in the treatment of skin, skin disorders and in promotion of hair growth that utilize controlled free radical formation and/or mild chemical irritant to achieve dermal cell repair and follicle stimulation. Compositions of the instant invention comprise novel, synergistic combinations of exfoliating agents, peroxidant reducing agents, and trace metal catalysts. These compositions may be used to provide useful improvements in hair growth, skin improvement (in condition, tone and appearance), to treat wounds, skin inflammation and for insect bite relief.

More specifically, preferred compositions of the present invention comprise a redox agent, preferably as an enediol-containing component such as an ascorbate derivative or dihydroxy maleic acid derivative, a dermatologically acceptable transition metal-containing component such as ferrous histidine, a carrier and optionally, a dermatologically active enzymatic component such as coenzyme CoQ10 (which may be used as a component in certain hair care formulations of the present invention) and optionally a desquamation/exfoliating agent, preferably as a dermatologically acceptable acid or ester. In compositions of the instant invention, the redox agent, preferably as an enediol-containing component such as an ascorbate derivative or other redux agent such as dihydroxymaleic acid, undergoes an oxidation reaction with the transition metal-containing component to produce hydrogen peroxide and enhance dermal health and hair growth. In alternative embodiments, an effective amount of a topical fever-producing agent and/or chemical irritant can be used in the present compositions in place of (i.e., as a replacement for) or in addition to the redox agent and the transition metal-containing component.

As stated, compositions of the instant invention optionally contain a dermatologically active acid as a desquamation/exfoliating agent that may be a cosmetically active acid or a pharmaceutically active acid, such as, for example, a hydroxy acid, ascorbic acid or a derivative thereof, lipoic acid, dihydrolipoic acid, or a combination thereof.

Compositions of the instant invention provide a visible improvement in skin condition shortly following application of the composition to the skin. Such improvement involves decrease in redness or swelling in dry or inflamed skin, improvements to skin imperfections such as textural discontinuities (including those associated with skin aging, such as age spots and keratoses) and other imperfections, and enhancing skin tone or color. In addition, compositions according to the present invention may be used to improve damaged or irritated skin. Compositions according to the present invention may also be used to promote wound healing or to treat skin inflammation or insect bites. Significantly, application of compositions of the instant invention to the human scalp induced hair growth.

In a preferred embodiment, the invention provides an improved skin care composition comprising lipoic acid, ascorbyl palmitate, an exfoliant cream base, ferrous histidine and optionally, coenzyme CoQ10.

The invention is described further in the following detailed description.

The present invention represents an unexpected result in that conventional dermatological sciences counsels for the use of antioxidants as anti-aging agents to avoid free radical formation, whereas the present invention relies on the formation of controlled free radical reactions that produce peroxide for much of its intended effect of promoting dermal stimulation and hair growth.

### DETAILED DESCRIPTION OF THE INVENTION

As used herein, the following terms have the following respective meanings. The term “dermatologically-acceptable,” as used herein, means that the compositions or components thereof so described are suitable for use in contact with human skin without undue toxicity, incompatibility, instability, allergic response, and the like. “Dermatologically active enzymatic component” includes antioxidant transducers of mitochondrial oxidative phosphorylation such as CoQ10. CoQ10 (coenzyme Q10, ubiquinone 50, 2,3-dimethoxy-5-methyl6-pentacontdacaenyl-benzoquinone) plays a vital role as a rate-limiting carrier for the flow of electrons through the mitochondrial complexes I, II and III of the respiratory chain, thereby maintaining or improving energy (ATP) generation by the mitochondria. It is also a major lipophilic antioxidant. The molecule is located in the inner mitochondrial membrane but is also associated with the membrane of other intracellular organelles. CoQ10 thus maintains redox activity and electron flow across different membranes (Villalba, Crane) and guarantees optimal mitochondrial functioning. The term “H₂CoQ₁₀” refers to the reduced form of CoQ₁₀, otherwise known as ubiquinol.

“Redox agents” or “redox agents that produce peroxide” are agents which produce hydrogen peroxide in the present invention. Exemplary redox agents include ascorbic acid (as well as ascorbate and ascorbate esters and other ascorbate derivatives and salts as described in greater detail herein) and dihydroxy maleic acid (which is preferred and may include esterified forms), among other compounds, especially those compounds which contain an enediol moiety, as set forth below.

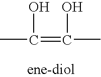

Ascorbic acid and derivatives thereof may be used as redox agents in the present invention. Ascorbic acid derivatives suitable for use in the instant invention include, but are not limited to, magnesium ascorbyl phosphate; sodium ascorbyl phosphate; sodium ascorbate; and ascorbyl glucosides. Ascorbic acid and derivatives thereof useful in the invention include, but are not limited to, ascorbyl caprilate, ascorbyl monoate, ascorbyl undeconate, ascorbyl laurate, ascorbyl trideconate, ascorbyl myristate, ascorbyl pentadeconate, ascorbyl palmitate, ascorbyl heptadecanate, ascorbyl stearate, ascorbyl monodecanate, and ascorbyl arachidate. Ascorbic acid and derivatives thereof useful in the invention also include metallic salts of ascorbic acid, including but not limited to sodium, calcium, and magnesium salts.

Preferred enediol-containing components include ascorbate derivatives and salts such as ascorbic acid-2-sulphate dipotassium salt, ascorbic acid-2-phosphate sequimagnesium salt, ascorbic acid-2-polyphosphate sequimagnesium salt and ascorbic acid-2-sulfate-tin. Note that these enediol containing compounds may also serve in certain instances as dermatologically acceptable acids or esters (desquamation/exfoliating agents) in the present compositions and methods. The inclusion of dihydroxymaleic acid or its pharmaceutically acceptable salt forms is preferred in certain embodiments according to the present invention. “Desquamation/exfoliating agents” are agents which are optionally included in compositions according to the present invention and enhance the skin appearance benefits of the present invention. They set in motion in an accelerating way, the skin's exfoliating process. It is a specific area of attack that sets up cell alarm signals in the dermal (including hair) processes to repair damage that is occurring. Hence, anything that affects the outermost layer of the dermis as part of the exfoliating process is contemplated for use in the instant invention. For example, the desquamation agents tend to improve the texture of the skin (e.g., smoothness). A variety of desquamation agents are known in the art and are suitable for use herein, including organic hydroxy acids (including alpha and beta hydroxy acids) such as salicylic acid, glycolic acid, lactic acid, 5-octanoyl salicylic acid, hydroxyoctanoic acid, hydroxycaprylic acid, and lanolin fatty acids. One desquamation system that is suitable for use herein comprises sulphydryl compounds and zwitterionic surfactants. Another desquamation system that is suitable for use herein comprises salicylic acid and zwitterionic surfactants. Additional exfoliating agents include, for example, protease or peptase enzymes (natural and bio-engineered) as well as other polypeptide compositions well-known in the art, bio-mimetic compounds that mimic alpha hydroxyl acids and include peptides, synthetic compounds that cut proteins successfully, and bioactive metals such as manganese, tin and copper (which may be included for its exfoliating properties quite separate from its metal catalysis characteristics), as well as natural soy-based products, such as those in the Johnson & Johnson Aveeno™ product line.

Quite unexpectedly, compositions according to the present invention produce smooth skin texture, which appears to be facilitated through additional growth and repair mechanisms (which appears to be induced by the removal of the outer layer of skin), and which are stimulated by the production of hydrogen peroxide. Thus, it is a combination of the exfoliating agents plus hydrogen peroxide signaling of cellular growth and repair mechanisms which represents an important aspect of the present invention which relates to skin treatment.

The term “dermatologically acceptable acid or ester” refers to certain desquamation/exfoliating agents and includes hydroxy acids such as alpha- or beta-hydroxy acids, poly-hydroxy acids, or any combinations of any of the foregoing. Preferably, the hydroxy acid is an alpha-hydroxy acid. Examples of alpha hydroxy acids include, but are not limited to, glycolic acid, lactic acid, malic acid, tartaric acid, pyruvic acid, citric acid, or any combination of any of the foregoing. Alpha hydroxyl acids are preferred in certain compositions for their ability to stimulate dermal cells to produce collagen and fibrinogen. Beta-hydroxy acids include, but are not limited to, salicylic acid. Lipoic acid and dihydrolipoic acid may also be used as dermatologically acceptable acids or esters.

A “dermatologically acceptable transition metal-containing component” includes compositions containing copper, iron, cobalt, manganese or tin such as copper histidine, iron (ferrous) histidine, ferrous EDTA, and copper EDTA and iron (ferrous) desferrioxamine and can include other salts such as the chloride, sulfate, (e.g. ferrous ammonium sulfate), nitrate and lactate salts of these metals, among others, including chelated complexes, as described below. Transition metals that are particularly preferred include Cu⁺², Cu⁺, Fe⁺², Fe⁺³, and Co⁺², as discussed above, preferably as chelates. Without intending to be bound to or limited by any theory, it is believed that transition metals are a key element in promoting beneficial controlled free radical production in the formulations of the instant invention. The reaction of ascorbate derivatives with transition metals favors beneficial hydrogen peroxide production.

In a preferred embodiment of the instant invention, the dermatologically acceptable transition metal-containing component is complexed with chelating agents such as EDTA, lactate, desferrioxamine, ethylene diammonium sulfate and tripeptide (diglycyl-1-histidine). Another set of chelates which may be used in the present invention are ferrous O-trensox and and ferric O-trensox, which are hydroxyquinoline-based iron chelators, which do not catalyze so-called Fenton reactions which produce biologically damaging hydroxyl radicals. See, J. Am. Chem. Soc., 117, 9760 (1995). The use of iron EDTA represents a preferred embodiment. Use of such chelating agent complexes provides beneficial reaction control in free radical production. Iron chelators, especially iron EDTA or iron desferrioxamine, are preferred for use in the present invention. Selective preferred chelators covering a range of ionization constants or having affinity constants ranging from about 10⁵ up to about 10⁵³ (even more preferably up to 10⁴³, within this range), are particularly useful for inclusion in the present invention.

In metal chelate-ascorbate systems, the histidine metal chelate causes only limited damage to DNA while EDTA chelates do not catalyze the Fenton reaction that can produce damaging OH radicals. It is believed that in these tight chelates there is insufficient metal ion available to decompose H₂O₂. No Fenton reaction is therefore possible and DNA scission is quenched. While less tightly bound transition metals with ascorbate are known to cause attack on DNA in vitro, recent work on the new biology of ascorbic acid demonstrates that in vivo systems containing ascorbate and transition metals, DNA is not attacked, as ascorbate acts as a protective agent.

Affinity Constants of Metal Chelates

| Chelate | Affinity Constant |
|---|---|
| Copper lacate | 10¹⁰-10¹² |
| Copper histidine | 10¹⁶ |
| Copper EDTA | 10²³ |
| Copper gluconate | 10⁶ |
| Ferrous lactate | 10¹² |
| Ferrous histidine | 10¹⁶ |
| Ferrous EDTA | 10²⁵ |
| Desferrioxamine | 10³⁰ |
| Ferric FDTA | 10²⁵ |
| Glycolic acid | 10⁵ |
| Copper tripeptide (GHG) | 10¹⁶ |
| Ferric salicylic acid | 10¹⁶ |
| Ferric catechol | 10²⁰ |

From the above, it is seen that the invention of selective catalytic metal activity can produce a variety of therapeutically beneficial results. All that is required is that the metal ion chelate have special arrangements that provide either full or limited access to O₂ and H₂O₂ and further that the ionization (or affinity) constant of the chelate be sufficient to control the specific end products of the reaction. Thus, the scope of the present invention includes a broad range (scope) of metal chelators to achieve various effects in dermal treatment.

In the present invention, “dermatologically acceptable chemical irritants” are optional components for use in the present invention. These agents also may be used as alternatives to redox agents and transition metal containing components in the present invention. These are agents which produce a measured and non-damaging skin irritation, at least a general reddening of the skin which is exposed to the agent and in certain instances, swelling and related physiological responses. These agents are known to produce a dynamic complex of cytologic and histologic reactions which occur in the affected blood vessels and tissues being exposed to these agents. The skin to which these agents are applied typically respond to these agents in local reactions and morphological changes, destruction or removal of the irritant from the tissue, and responses which lead in the tissue to repair or healing. The irritation which occurs from these agents and the physiological response to that irritation is advantageously used in the present invention. These agents may be used in addition to, or instead of (i.e., as replacements for) redox agents/transition metal-containing components in the present invention. Examples of such agents include various proteolytical enzymes as well as other enzymes, alcohol, including grain spirits or rubbing alcohol (isopropanol), ammonia spirit, aromatics, creosite, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh, and topical mild fever agents as described below, among others.

“Topical mild fever agents” are those agents which fall under the rubric of dermatologically acceptable chemical irritants and induce a very mild topical local fever in skin which may be used to further promote stimulation of skin and/or hair follicles in the present invention. Although not considered exfoliating agents, these agents are similar to exfoliating agents in that they stimulate the skin for further growth, often, however, without attacking cells (in the dermal layer) from the skin. These agents produce mild elevation of skin temperatures and pyrogens. These agents include, for example, capsaicin, piperine, mustard, nicotinic acid, camphor, menthol, among others agents or irritants. These agents may be used in addition to, or instead of (i.e., as replacements for) redox agents and transition metal containing component.

The term “wound” means a superficial or topical wound of the skin such as a burn, cut, scrape, scratch, minor irritation or surgical wound. The term inflammation means inflammation of the skin, whether that irritation is considered a wound or is simply considered damaged skin. “Damaged skin” is skin which is has been sunburned, contains dermal lesions, irritation or imperfections which do not rise to the level of a wound and can include wrinkles and other conditions which exist as a consequence of natural processes, including aging, exposure to the sun, etc.

The term “skin smoothness” is used to refer to tactile skin properties that encompass one or more of the following: roughness, suppleness, elasticity, softness, friction, dryness, scaling, and pliability. In certain embodiments, compositions according to the present invention enhance the smoothness of skin, including damaged skin.

“Carriers” include compositions suitable for topical application to the skin within which the essential materials and optional other materials are incorporated to enable the essential materials and optional components to be delivered to the skin at an appropriate concentration. The carrier can thus act as a diluent, dispersant, solvent, or the like for the various components of the instant compositions including particulate material(s) and the actives which ensure that they can be applied to and distributed evenly over the selected target at an appropriate concentration.

The carrier can be solid, semi-solid or liquid. Highly preferred carriers are liquid or semi-solid, such as creams, lotions and gels. Preferably, the carrier is in the form of a lotion, cream or a gel, more preferably one which has a sufficient thickness or yield point to prevent the particles from sedimenting. The carrier can itself be inert or it can possess dermatological benefits of its own. The carrier should also be physically and, chemically compatible with the essential components described herein, and should not unduly impair stability, efficacy or other use benefits associated with the compositions of the present invention. Preferably, active components are micronized for inclusion into the compositions for enhanced activity.

The type of carrier utilized in the present invention depends on the type of product form desired for the composition. The topical compositions useful in the subject invention may be made into a wide variety of product forms such as are known in the art. These include, but are not limited to, lotions, creams, gels, sticks, sprays, ointments, pastes, and mousses. These product forms may comprise several types of carriers including, but not limited to, solutions, aerosols, emulsions, gels, solids, and liposomes. Preferred carriers contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), polypropylene glycol (e.g. of MW 425-2025), glycerol, butylene glycol, 1,2,4-butanetriol, sorbitol esters, 1,2,6-hexanetriol, ethanol, iso-propanol, sorbitol esters, ethoxylated ethers, propoxylated ethers and combinations thereof. The diluent is preferably liquid. Water is an especially preferred diluent. The composition preferably comprises at least about 60% of the hydrophilic diluent.

Preferred carriers comprise an emulsion comprising a hydrophilic phase, especially an aqueous phase, and a hydrophobic phase e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 99% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. Preferred compositions herein are oil-in-water emulsions.

“Therapeutically effective amount” as used herein means an amount of a composition of the instant invention that, when applied to the skin of a mammal, moisturizes the skin, reduces irritation, enhances skin tone, reduces wrinkles, reduces scaling, inhibits or otherwise treats inflammatory disorders including psoriasis, stimulates skin cell growth, or stimulates hair follicles or hair growth.

The term “effective amount” subsumes the term therapeutically effective amount within it and is directed to an amount of a composition, compound or component which produces an intended effect within the context of its use, whether that use is for the improvement in skin, hair growth, treatment of wounds, treatment of inflammation and insect bite relief. Of course, the final use of the composition may affect the amount of agent to be included within a particular formulation or composition. For example, skin treatment formulations, in contrast to hair growth formulations, would have reduced amounts of CoQ10 or other enzymatic active component, or in some cases, even eliminate this component. Wound healing formulations would emphasize the inclusion of redox agent along with the transition-metal containing component.

Note that hair care formulations may be formulated to increase follicle cell growth (increase the size and activity of hair follicles) without producing irritation of the surrounding skin. The following formula is relevant here:

| | |
|---|---|
| Eucerin ™ | 4.0 g. |
| Ascorbate solution (15%) | 1.0 cc |
| Iron EDTA (1%) solution | 1.0 cc |
| EDTA Solution | 2 Drops |

The above formula in a skin test showed a multiplicity of very small, pin-head like projections on the epidermis produced by activation of the papilla- corium layer. Papilla have blood vessels and nerves interlaced. They nourish every hair follicle. The activation ensues for about 2-4 hours and is from slight to gross depending on the formulation and returns to the normal skin landscape. The bioreactivity of the formula can be judged by the intensity and time of the reaction. The above shows that it is possible to have skin bioreactivity by a formulation that does not cause free radical damage to DNA.

Note that an alternative formula which may be preferred includes Eucerin™ 9.6 g, ascorbate solution (15% solution of the sodium salt) of 0.2 cc and 0.2 cc of a 1.0% by weight ferrous EDTA solution.

The following table provides a general overview of individual components and their preferred weight ranges in preferred compositions according to the present invention. Note that in some circumstances, certain components may be optional as otherwise disclosed herein. One of ordinary skill in the art may readily modify the type and amount of components as otherwise taught herein in practicing the present invention.

TABLE 1
Recommended Concentrations-Weight %
of Individual Components

| | Wound | Skin | Hair |
|---|---|---|---|
| CoQ10 | | | |
| range | 0.5-15 | 0-2 | 0.1-10 |
| preferred | 1-3 | 0.1-0.5 | 0.5-1.5 |
| optimum | 2 | 0.3 | 2 |
| Redox Agent | | | |
| range | 0.5-10 | same | same |
| preferred | 0.5-2 | same | same |
| optimum | 2.5 | same | same |
| Metal Salt | | | |
| range | 0.001-5 | 0.001-5 | 0.001-5 |
| preferred | 0.01-1 | 0.01-0.3 | 0.01-0.3 |
| optimum | 0.5 | 0.05 | 0.05 |
| Exfoliating Agent | | | |
| range | 0-15 | 0.5-15 | 0.1-15 |
| preferred | 3-12 | same | same |
| optimum | 10 | same | same |

| | |
|---|---|
| Skin Irritant | Broad range of amount from micro to macro |
| | depending on particular Agent and activity. |
| | The range is extremely large and preferably is |
| | from about 0.001% to about 10%. |

The term “exfoliant cream base” refers to a cream base or lotion which comprises on a weight/weight basis about 2% to about 20% (preferably, about 5% to about 15%, more preferably about 10%) by weight of a desquamation/exfoliating agent, preferably an alpha hydroxy acid, more preferably glycolic, lactic acid or a dermatologically acceptable salt; about 2% to about 20% by weight of a plasticizing agent, preferably urea, in a preferred amount ranging from about 5% to about 15%, more preferably about 10% and a standard topical cosmetic/pharmaceutical lotion or cream base making up about 60% to about 96%, more preferably, about 65% to about 93%, even more preferably about 80% of the exfoliant cream base.

The topical compositions of the present invention may comprise a wide variety of optional components, provided that such optional components are physically and chemically compatible with the essential components described herein, and do not unduly impair stability, efficacy or other use benefits associated with the compositions of the present invention. Optional components may be dispersed, dissolved or the like in the carrier of the present compositions. Optional components include emollients, oil absorbents, antimicrobial agents, binders, buffering agents, denaturants, cosmetic astringents, external analgesics, film formers, humectants, opacifying agents, perfumes, pigments, skin soothing and healing agents, preservatives, propellants, skin penetration enhancers, solvents, suspending agents, emulsifiers, cleansing agents, thickening agents, solubilising agents, waxes, sunscreens, sunless tanning agents, antioxidants and/or radical scavengers, chelating agents, anti-acne agents, anti-inflammatory agents, desquamation agents/exfoliants, organic hydroxy acids, vitamins and natural extracts. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker,. Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993) can also be used in the present invention.

A particularly preferred antioxidant for use in the present invention is alpha lipoic acid, or its pharmaceutically acceptable salt form, preferably in its reduced form, which may be included as a defoliation/desquamation agent or separately, for its beneficial characteristics as an antioxidant. This component may be added in amounts ranging from about 0.005% to about 10.0%, more preferably about 0.01% to about 1% by weight (when used as an antioxidant as opposed to its alternative use as an exfoliating/desquamation agent) for its beneficial antioxidant effects on cells, which may provide benefit in the cellular growth and repair mechanisms which are facilitated by compounds according to the present invention.

A safe and effective amount of a desquamation/exfoliating agent may be added to the compositions of the subject invention, more preferably from about 0.1% to about 20%, even more preferably from about 0.2% to about 10%, also preferably from about 0.5% to about 4% of the composition. In addition, agents which induce a very mild topical local fever in skin (“topical mild skin agitants”) such as pepper (capsaicin), piperine, mustard, nicotinic acid, camphor and menthol, among others, may also be used in place of, or in addition to, the desquamation agent or exfoliant, essentially the same amount as the desquamation/exfoliating agent.

Compositions according to the present invention may also include a peptide such as a tripeptide, alone or in combination with a metal such as a copper (II) or tin (II) chelate of the tripeptide Gly-L-His-L-Lys and other peptides or additives which are known to enhance wound healing and to otherwise improve attributes of skin.

The pH range of compositions of the instant invention is approximately 4-9, more preferably 5-7, and even more preferably about 6-7.

The invention is described further in the following examples, which are illustrative and not limiting. All percentages, parts and ratios are by weight of the total composition, unless otherwise specified. All such weights as they pertain to listed ingredients are based on the specific ingredient level and, therefore, do not include solvents, carriers, by-products, filler or other minor ingredients that may be included in commercially available materials, unless otherwise specified.

### EXAMPLE 1

Hair Cream

A hair cream of the instant invention is prepared by mixing the following components in the designated weight percentages with an appropriate carrier through dermatological formulation techniques that are well-known in the art. The illustrated hair cream can be formulated at room temperature and atmospheric pressure and the resulting reactions are readily controlled to yield the desired composition.

| Component | Weight Percentage |
|---|---|
| *CoQ10 (submicron) | 7 |
| Lipoic Acid (micronized) | 3 |
| Ascorbyl palmitate | 3 |
| **Exfoliant cream base | 86.5 |
| Ferrous histidine | 0.5 |

*Bio-Qt ™ Essentials-From Julian Whitaker, Healthy Directions-in 70% by weight soybean oil (5% of composition weight is soybean oil and 2% of composition weight is CoQ10).
**Exfoliant cream base is made of (w/w) 10% by weight lactic acid partial salt, 10% by weight urea and 80% of a standard topical cosmetic/pharmaceutical lotion or cream.

The hair cream of this example may be applied to the skin of a mammal for enhancement of hair growth. Application of the skin cream to the scalp of a mammal in a concentration of between about 0.3 to 0.5 gm/cm² twice daily, may result in hair growth stimulation over a period of about 3 to 4 months.

### EXAMPLE 2

Gel

A gel of the instant invention is prepared by mixing the following components in the designated weight percentages with an appropriate carrier through dermatological formulation techniques that are well-known in the art. The illustrated gel can be formulated at room temperature and atmospheric pressure and the resulting reactions are readily controlled to yield the desired composition.

| Component | Weight Percentage |
|---|---|
| CoQ10 (micronized) | 2 |
| Lipoic Acid (micronized) | 3 |
| Ascorbyl palmitate | 3 |
| Exfoliant gel base | 88.5 |
| Ferrous histidine | 0.5 |
| Soybean oil | 3 |

The gel of this example may be applied to the skin of a mammal for enhancement of skin health and condition and pH changes and skin tone and color may be monitored. Application of the gel to the scalp of a mammal in a concentration of between about 0.3 to 0.5 gm/cm² twice daily may result in hair growth stimulation over a period of 3 to 4 months.

### EXAMPLE 3

Lotion

A lotion of the instant invention is prepared by mixing the following components in the designated weight percentages with an appropriate carrier through dermatological formulation techniques that are well-known in the art. The illustrated lotion can be formulated at room temperature and atmospheric pressure and the resulting reactions are readily controlled to yield the desired composition.

| Component | Weight Percentage |
|---|---|
| CoQ10 (Solubilized)* | 2 |
| Lipoic Acid (micronized) | 3 |
| Ascorbyl palmitate | 3 |
| Exfoliant lotion base | 91.5 |
| Copper histidine | 0.5 |

*Q-Gel ™ 100 From Tishcon, Crop., Westbury, New York.

The lotion of this example may be applied to the skin for enhancement of skin health and condition and pH changes and skin tone and color may be monitored. Application of the lotion to the scalp in a concentration of between about 0.3 to 0.5 gm/cm² twice daily may result in hair growth stimulation over a period of 3 to 4 months.

### EXAMPLE 4

Ointment

An ointment of the instant invention is prepared by mixing the following components in the designated weight percentages with an appropriate carrier through dermatological formulation techniques that are well-known in the art. The illustrated ointment can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Ascorbyl palmitate | 3 |
| Exfoliant ointment base | 91.5 |
| Copper histidine | 0.5 |

The ointment of this example may be applied to the skin of a mammal for enhancement of skin health and condition and pH changes and skin tone and color may be monitored. Application of the lotion to the scalp of a mammal in a concentration of between about 0.3 to 0.5 gm/cm² twice daily may result in hair growth stimulation over a period of 3 to 4 months.

### EXAMPLE 5

Skin Cream

A skin cream of the instant invention is prepared by mixing the following components in the designated weight percentages through dermatological formulation techniques that are well-known in the art. The illustrated skin cream can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Dihydroxymaleic acid | 1 |
| Exfoliant cream base | 91.5 |
| Ferrous histidine | 0.5 |

The skin cream of this example may be applied to the skin of a mammal for enhancement of skin health and condition and pH changes and skin tone and color may be monitored. Application of the skin cream to the scalp of a mammal in a concentration of between about 0.3 to 0.5 gm/cm² twice daily may result in hair growth stimulation over a period of 3 to 4 months.

### EXAMPLE 6

Skin Cream

A skin cream of the instant invention is prepared by mixing the following components in the designated weight percentages with an appropriate carrier through dermatological formulation techniques that are well-known in the art. The illustrated skin cream can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Ascorbyl palmitate | 3 |
| Exfoliant cream base | 91.5 |
| Copper EDTA | 0.5 |

The skin cream of this example may be applied to the skin of a mammal for enhancement of skin health and condition and pH changes and skin tone and color may be monitored. Application of the skin cream to the scalp of a mammal in a concentration of between about 0.3 to 0.5 gm/cm² twice daily may result in hair growth stimulation over a period of 3 to 4 months.

### EXAMPLES 7A and B

Skin Creams

Skin creams of the instant invention are prepared by mixing the following components in the designated weight percentages with an appropriate carrier through dermatological formulation techniques that are well-known in the art. The illustrated skin creams can be formulated at room temperature and atmospheric pressure and the resulting reactions are readily controlled to yield the desired composition.

| | |
|---|---|
| Component | Weight Percentage |

Composition A

| | |
|---|---|
| CoQ10 | 2 |
| Lipoic Acid (micronized) | 3 |
| Ascorbyl palmitate | 3 |
| Exfoliant cream base | 91.5 |
| Ferrous EDTA | 0.5 |

Composition B

| | |
|---|---|
| CoQ10 | 2 |
| Lipoic Acid (micronized) | 3 |
| Ascorbyl palmitate | 3 |
| Exfoliant cream base | 91.9 |
| Ferrous ethylenediammonium sulfate | 0.1 |

The skin cream(s) of this example may be applied to the skin of a mammal for enhancement of skin health and condition and pH changes and skin tone and color may be monitored. Application of the skin cream to the scalp of a mammal in a concentration of between about 0.3 to 0.5 gm/cm² may result in hair growth stimulation over a period of 3 to 4 months.

### EXAMPLE 8

A skin care preparation was made by incorporating the following:

| | |
|---|---|
| CoQ₁₀ | 2% |
| Lipoic Acid | 3% |
| Ascorbyl palmitate | 5% |

into a skin cream. The mixture was transferred to aluminum metal tubes for dispensing. This mixture was shown to grow hair in modest amounts. However, when this formulation was used at the rate of six (6) times per day, astoundingly, it caused hair to fall out. From this experiment, we concluded that there was some growth promoter in the formulation that caused hair growth if used in reasonable amounts.

### EXAMPLE 9

The above experience of Example 8 caused us to question the ascorbyl component as ascorbate is known to act as a prooxidant under some conditions. We therefore turned to more simple systems as follows:

| | |
|---|---|
| Co Q₁₀ | 2% |
| Lipoic Acid | 3% |

dissolved in a semiliquid ester of fatty acids. This formulation series grew an abundance of hair. Unforunately, the hair that grew was all vellus hair. We were unable to convert this very short, thin vellus hair to long terminal hair. We concluded that Co Q₁₀ exhibited extraordinary ability to cause dormant follicles to grow hair but more was needed in activation to grow terminal hair.

### EXAMPLE 10

The next formulation contained the dihydro or reduced form of CoQ₁₀ (uibiquinol) as follows:

| | |
|---|---|
| Reduced Co Q₁₀ (Ubiquinol) | 2% |
| Lipoic Acid | 3% |
| Ascorbyl palmitate | 5% |

The skin cream lotion used in the formulation containing the above ingredients contained an exfoliating agent (lactic acid). This combination was transferred to aluminum dispensing tubes.

This composition was beginning to show some activity after only one month and after 3 months it was considered to be a most remarkable hair grower. We concluded that the high bioavailability of Co Q₁₀ in the reduced form (water soluble) was responsible for the improved results. Also, the exfoliating mechanism, induced by the hydroxy acid (lactic acid), was a contributing factor.

### EXAMPLE 11

A fresh formulation of example 10, above, was put into a glass container (1) and aluminum metal tubes (2). Sample 2 was very substantially better. It was concluded that the aluminum dispensing tube played a definite part in the successful hair growth program- a quite unexpected result.

### EXAMPLE 12

The two formulations of example 11, above were put under observation. It was observed that on standing, pressure and odor developed in sample (2). More samples were made from this type of formulation using varied exfoliating creams. The gas was identified as hydrogen (H₂) and the more acidic the formulation the more gas was produced until one acted like a geyser. It was concluded that lactic acid attacked the aluminum tubes and the vigor depended on acidity. It was also concluded that the metals of that alloy are important and appear to be a significant factor in the hair growth exhibited. It was therefore concluded that trace metal catalysts are an important component of the present invention.

### EXAMPLE 13

The purpose of this example is to demonstrate the use of dihydroxy-maleic acid as an enediol compound. (extension of ascorbic acid as part of a generic class).

The dihydroxy maleic acid DHM was neutralized with NaOH to pH 6.5 and then prepared to a 15% solution.

| | | |
|---|---|---|
| Eucerin ™ | 7.9 | g. |
| DHM (15%) | 1.1 | c.c. |
| Fe EDTA (1%) | 1.0 | c.c. |

The catalyst Fe EDTA was prepared as a 1% solution of Mohr's salt Fe(NH)₄SO₄.6H₂O) and a molar amount of ethylene diamine tetra acetic acid or Fe EDTA.2Na. Then 10% extra EDTA was added to the final catalyst solution.

Results

The formulation produced excellent bioactivity on my skin test. No hair growth tests were made as animal studies are necessary. This composition does not cause DNA scission.

### EXAMPLE 14

Purpose

a) To use Atrac-Tain * as a vehicle.

b) To use sodium ascorbate in place of ascorbyl palmitate

| | | |
|---|---|---|
| Atrac-Tain | 8.0 | g. |
| Soybean oil | 1.1. | g. |
| Sodium ascorbate (1.5%) | 1.0 | cc. |
| CoQ₁₀ (micronized) | 0.2 | g. |
| Copper lactate (0.5%) | 1.0 | cc. |

*contains alpha hydroxy acid

The copper lactate solution was prepared by adding 0.5% copper sulfate pentahydrate to a molar quantity of lactic acid and then pH adjusted to 6.3 with NH₄OH.

Results

The homogenized mix with soybean oil as a carrier for CoQ₁₀ was very stable. It diffused into the skin on application as the CoQ₁₀ orange color disappeared.

It produced an excellent bio-reactive effect in my new skin test.

The sodium ascorbate successfully replaced ascorbyl palmitate.

The formulation is similar to the one used to grow hair on animals.

### EXAMPLE 15

Formulation (2) was diluted with Atrac-tain in a 1 to 4 ratio.

Results

The color of the above formulation is excellent and it is still bioactive. Cu lactate is an excellent chelate form for bioactivity (see 14 also)

### EXAMPLE 16

Purpose

To use copper histidine as a catalyst with sodium ascorbate.

There is no CoQ₁₀ in the formulation.

| | |
|---|---|
| Eucerin ™ | 9.72 |
| Copper Histidine* 1% | 0.03 cc |
| Ascorbate (1.5%) | 0.25 cc. |

One drop of saturated Histidine solution
is added to the formulation.

Results
Color of mix very good and bioactivity on skin was demonstrated.
This example as with example (1) depends on the copper-(or ascorbate
reaction catalyst)
(1)) ascorbate reaction catalyst for it's effect on skin.
*The metal catalyst Cu Histidine was prepared by adding 1% Copper Sulfate penta-hydrate to a molar quantity of Histidine and then adjusting the pH to 6.3. The salt is Cu Histidine.

### EXAMPLE 17

| | |
|---|---|
| Atrac-Tain | 4.8 |
| Ascorbate 1.5% | 0.1 c.c. |
| Fe EDTA (1%) | 0.1 c.c. |

Results

Long Term color evidenced on spot plate. Outstanding bioreactivity is very satisfactory on skin.

Note: Another metal-ascorbate reaction catalyst only system.

### EXAMPLE 18

| | |
|---|---|
| Aveeno (J & J Cream) | 4.8 |
| Ascorbate (15%) | 0.1 cc |
| Fe EDTA (1%) | 0.1 cc |

Add one drop of EDTA saturated solution and allow to equilibriate for 2 days.

Results

Initial color excellent, but on aging, it was not as good. If higher concentrations of ascorbate-iron EDTA catlyst are used the Aveeno mix, on aging, turns dark brown or even black.

Note: There is no alpha hydroxy acid in the formula- just iron-ascorbate reaction catalyst, yet bioactivity was quite good.

### EXAMPLE 19

Dilute the Aveeno formulation in example (18) in a 1-1 ratio with Aveeno Cream.

Results

Very definite action, might be sufficient for commercial use even at this high dilution. (1-10×).

Only ascorbate-iron catalyst at work and free radical action is below DNA scission for this reaction. (as in example 18 with Aveeno).

In Summary.

Examples 13-19 show the following:

1. Sodium ascorbate can be used as a substitute for ascorbyl palmitate.

2. Dihydroxymaleic acid in place of ascorbate is another example of the enediol generic class.

3. Iron and Copper were chelated so tightly that DNA scission no longer occurs still shows bioreactivity on skin when combined with ascorbate (or an enediol). Iron is definitely the more active catalyst metal.

4. CoQ₁₀ and Alpha hydroxy acids are not necessary for activation of the bioreactivity measurement on skin. Hair growth is unknown.

5. Soybean oil is an excellent transporter of CoQ₁₀ across the skin barrier.

6. J & J Aveeno, Daily Moisturizing Lotion, is an acceptable vehicle for our ascorbate metal reaction catalyst, provided the catalyst is used in the more dilute range.

7. Tightly bound Copper Histidine is an acceptable metal catalyst for the ascorbate metal reaction catalyst. The chelated but somewhat less tightly bound copper in copper lactate is a preferred catalyst based on our skin studies as well as hair growth in humans and animals.

Discussion of Reaction Mechanisms

To date we have based our case on the generation of hydrogen peroxide by our own enediol-trace transition metal catalyst. It is believed that the hydrogen peroxide as so produced with the use of our dermal formulation produces cellular signaling. Hydrogen peroxide is a well known cell signaling agent. This activates innate growth and repair mechanisms which result in skin improvement and hair growth. Hence, the complex molecular biology of dermal cells is sent into motion by a simple molecule, hydrogen peroxide. The intensity of the bioactivation is readily controlled by the amount of enediol as well as the metal chelates. The metallic ions of iron or copper are under strict control by the choice of chelating agent. The control is so good that specific reactions can be quenched like DNA scission while allowing the core growth and repair function to continue.

Possible Additional Reactions

Examination of example 18 shows excessive reactivity even using tight metal chelation. It could be that in some cream bases there are components that can be reactive in the presence of our ascorbate-iron catalyst. The inventors were able to control this. This suggests that the enediol -metal catalyst is doing more than produce hydrogen peroxide. The ascorbate- metal catalysis in a skin cream that grew hair 5 years ago for us, was a discovery that is still unfolding as to how it works. Example 6 is suggestive of the possibility that ascorbate-metal, as a reaction catalyst, may be a factor in the hair growth we observed.

### EXAMPLE 20

The following composition was prepared and tested in laboratory test animals:

| Eucerin ™ Renewal* | 33.00 g |
|---|---|
| Ascorbyl Palmitate | 1.60 g |
| Copper Lacate | 1.5 cc of a 1% solution |
| Coenzyme Q | 0.6 g |
| Soybean Oil | 1.15 |

*Eucerin ™ Renewal from Beiersdorf, Inc. Wilton, Connecticut, USA.
Copper lactate is 1% salt, copper sulfate penta hydrate with a molar quantity of lactic acid.

The ingredients were thoroughly hand mixed for each material added followed by successive intermittent homogenization at high speed being careful not to overheat the emulsion. The ascorbyl palmitate was successfully dispersed for good bioavailability. Minutes to hours later the CoQ₁₀ spontaneously appeared to have reduced to the mostly reduced H₂CoQ₁₀. The CoQ10 was reduced with ascorbyl palmitate using vigorous agitation-the presence of soybean or other vegetable oil is helpful in the reduction reaction.

The hair growth composition as prepared above was evaluated in the C3H mouse model of hair growth, in parallel with the benchmark 2% Minoxidil. 6-week old female C3H mice were purchased from Taconic Labs and acclimated for 1 week. When all mice were confirmed to be in telogen, approximately 1.5×5 cm dorsal area of the mice was clipped and the test materials were applied over the clipped area once daily, with weekends off, for several weeks. A group of control mice that were clipped and left untreated was also included. Since a placebo cream was not provided, this study did not evaluate the effect of the placebo cream composition on hair growth.

Mice treated with the composition according to the present invention showed initial hair growth on the treated area at 2 weeks after the start of the treatment. Neither in the control nor the Minoxidil treated mice was visual hair growth seen at this time. The results were confirmed by histological analysis of Fontana-Mason stained sections of mouse skin. Hair follicles in the anogen phase of the hair cycle were observed in the mouse skin sections treated with the composition of the present invention, but not in the control or in the Minoxidil treated mice.

1. A composition comprising, in effective amounts:
(a) at least one redox agent that produces peroxide;
(b) a dermatologically acceptable transition metal-containing component;
(c) a carrier;
(d) optionally, a dermatologically active enzymatic component; and
(e) optionally, a desquamation/exfoliating agent, wherein the composition has a pH of approximately 4 to 9.

2. The composition of claim 2, wherein:
(a) the dermatologically active enzymatic component is an antioxidant transducer of mitochondrial oxidative phosphorylation;
(b) said redox agent is ascorbic acid, an ascorbate derivative or salt or dihydroxymaleic acid or a salt; and
(c) the dermatologically acceptable transition metal-containing component contains copper, iron, cobalt, or manganese; and
(d) the optional desquamation/exfoliating agent is a dermatologically acceptable acid or ester composition or polypeptide composition.

3. The composition of claim 2, wherein:
(a) the dermatologically active enzymatic component is CoQ₁₀ or H₂CoQ₁₀;
(b) the redox agent is an ascorbate ester or salt; and
(c) the dermatologically acceptable transition metal-containing component is copper histidine, ferrous histidine, ferrous EDTA, ferrous desferrioxamine, copper EDTA or mixtures thereof; and
(d) the desquamation agent is an alpha or beta hydroxy acid or mixtures thereof.

4. The composition of claim 3 wherein said acid is lactic acid, salicylic acid or mixtures, thereof.

5. The composition of according to claim 2, wherein the weight percentage ratio of redox agent to dermatologically acceptable transition metal-containing component is from approximately 10:1 to approximately 5:1.

6. The composition of claim 2, wherein the weight percentage ratio of redox agent to dermatologically acceptable transition metal-containing component is approximately 6:1.

7. A composition of claim 1 comprising by weight about 1% to about 10% CoQ10, about 1%-10% lipoic acid (micronized), about 1% to 10% ascorbyl palmitate, about 80% to 95% of an exfoliant cream base, and about 0.2% to 1.5% ferrous histidine.

8. A composition of claim 1 comprising by weight about 1% to about 10% CoQ10, about 1%10% lipoic acid (micronized), about 1% to 10% ascorbyl palmitate, about 80% to 95% exfoliant cream base, and about 0.2% to 1.5% ferrous EDTA.

9. A composition of claim 1 comprising by weight about 1% to about 10% CoQ10, about 1%-10% lipoic acid (micronized), about 1% to 10% ascorbyl palmitate, about 80% to 95% exfoliant cream base, and about 0.2% to 1.5% copper histidine.

10. A composition of claim 1 comprising by weight about 1% to about 10% CoQ10, about 1%-10% lipoic acid (micronized), about 1% to 10% ascorbyl palmitate, about 80% to 95% exfoliant cream base, and about 0.2% to 1.5% copper lactate.

11. A composition of claim 1 comprising by weight about 1% to about 10% CoQ10, about 1%-10% lipoic acid (micronized), about 1% to 10% dihydroxymaleic acid, about 80% to 95% exfoliant cream base, and about 0.2% to 1.5% ferrous histidine.

12. A composition of claim 1 comprising by weight about 0.2% to 2.0% CoQ₁₀, about 1% to about 10% lipoic acid, about 80% to about 90% of an exfoliant cream base and about 0.001% to about 0.01% ferrous histidine.

13. A composition of claim 1 comprising by weight about 0.2% to 2.0% CoQ₁₀, about 1% to about 10% micronized lipoic acid, about 1% to about 5% ascorbyl palmitate, about 1% to about 5% soybean oil, about 80% to about 95% of an exfoliant cream base and about 0.01% to about 0.2% ferrous histidine.

14. A composition of claim 3 wherein:
1. the dermatologically active enzyme component is H₂CoQ₁₀;
2. the redox agent is an ascorbate derivate or salt; and
3. the dermatologically acceptable transition metal-containing component is copper histidine, ferrous histidine, ferrous EDTA, ferrous desferrioxamine or copper lactate.

15. A composition of claim 3 wherein the CoQ₁₀ is submicronized.

16. A composition of claim 1 comprising about 0.1% to 2.0% CoQ₁₀, about 1% to about 10% lipoic acid in micronized form, about 1% to about 10% ascorbyl palmitate, about 1% to about 5% fatty acid transport oil and about 0.01% to about 0.1% ferrous histidine.

17. A composition according to claim 1 in topical dosage form.

18. The composition according to claim 1 wherein said dosage form is a skin cream, lotion, emulsion or gel.

19. A composition according to claim 1 including an effective amount of a chemical irritant in place of or in addition to said redox agent and said transition-metal containing component.

20. The composition according to claim 19 wherein said chemical irritant is selected from the group consisting of ethanol, isopropanol, ammonia spirit, aromatics, creosite, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh, capsaicin, piperine, mustard, nicotinic acid, camphor, menthol and mixtures thereof.

21. A composition according to claim 3 further including a chemical irritant selected from the group consisting of ethanol, isopropanol, ammonia spirit, aromatics, creosite, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh, capsaicin, piperine, mustard, nicotinic acid, camphor, menthol and mixtures thereof.

22. A method of treating an inflammatory disorder of the skin comprising administering to a mammal in need thereof a therapeutically effective amount of a composition according to claim 1.

23. A method of stimulating mammalian skin follicles comprising topically administering to a mammal a therapeutically effective amount of a composition according to claim 1.

24. A method of stimulating mammalian hair growth comprising topically administering to a mammal in an area of the skin or scalp where hair growth is to be stimulated a therapeutically effective amount of a composition according to claim 1.

25. A method of stimulating mammalian hair growth comprising topically administering to a mammal in an area of the skin or scape where hair growth is to be stimulated a therapeutically effective amount of a composition of claim 4.

26. The method of claim 21 wherein the mammal is a human and the composition is administered to the scalp.

27. The method of claim 22 wherein the mammal is a human and the composition is administered to the scalp.

28. The method of claim 23 wherein the mammal is a human and the composition is administered to the scalp.

29. A method of treating a wound in the skin of a mammal comprising applying to said wound an effective amount of a composition according to claim 1.

30. The method according to claim 29 wherein said wound is a bum, cut, scrape, scratch, minor irritation or surgical wound.

31. A method of treating damaged skin comprising applying to said skin an effective amount of a composition according to claim 1.

32. A method of treating skin to enhance its smoothness comprising applying to said skin a composition according to claim 1.

33. A composition comprising, in effective amounts:
1. a dermatologically active enzymatic component;
2. at least one chemical irritant;
3. a carrier; and
4. optionally, a desquamation/exfoliating agent, wherein the composition has a pH of approximately 4 to 9.

34. The composition according to claim 31 wherein said chemical irritant is selected from the group consisting of ethanol, isopropanol, ammonia spirit, aromatics, creosite, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh and mixtures thereof.
